(19) United States
(12) Patent Application Publication
(10) Pub. No.: US 2024/0008813 A1
(43) Pub. Date: Jan. 11, 2024

(54) SMART WEARABLE DEVICE AND METHOD FOR ESTIMATING TRADITIONAL MEDICINE SYSTEM PARAMETERS

(71) Applicant: AYUR.AI PRIVATE LIMITED, Chennai (IN)

(72) Inventor: Balasubrahmanyam Pesala, Chennai (IN)

(21) Appl. No.: 18/371,718

(22) Filed: Sep. 22, 2023

Related U.S. Application Data
(63) Continuation of application No. PCT/IB2022/052544, filed on Mar. 21, 2022.

(30) Foreign Application Priority Data
Mar. 22, 2021 (IN) 202141012087

(51) Int. Cl. A61B 5/00; A61B 5/024

(52) U.S. Cl. CPC A61B 5/4854; A61B 5/02427; A61B 5/486; A61B 5/746; A61B 5/681; A61B 5/7475; A61B 5/0002

(57) ABSTRACT

A wearable device for estimating traditional medicine system parameters is disclosed. The wearable device includes light sources, configured to stimulate skin of a patient through light rays. The wearable device includes sensors, configured to capture patient health parameters. The plurality of subsystems includes a medical input data collection subsystem configured to collect patient information, blood pulse parameters and the captured patient health parameters. The plurality of subsystems includes a health status computation subsystem, configured to apply the collected patient information the captured one or more patient health parameters and the blood pulse parameters onto trained machine learning model and estimate real time set of traditional medicine system parameters. The plurality of subsystems also includes a disease identification subsystem, configured to compare the real time set of traditional medicine system parameters with pre-stored traditional medicine system parameters, identify a disease and give recommendations.

### EARLIEST PRIORITY DATE

This application claims priority from a Provisional patent application filed in India having Patent Application No. 202141012087, filed on Mar. 22, 2021, and titled “SMART WEARABLE DEVICE AND A METHOD FOR AYURVEDA PRAKRITI AND VIKRITI ESTIMATION”.

### FIELD OF INVENTION

Embodiments of the present disclosure relates to a smart device, and more particularly to a smart wearable device and a method for estimating traditional medicine system parameters.

### BACKGROUND

Ayurveda, Siddha, Traditional Chinese Medicine and allied fields are holistic sciences that provide a unified framework for disease diagnosis and treatment. First step in Ayurveda diagnosis involves stratification of individuals according to their Prakriti (native state) and assessing Vikriti (diseased state) for realizing personalized and preventive treatments.

Prakriti estimation is typically done using a questionnaire. Vikriti estimation is very challenging as it depends on various complex factors such as age, sex, time of the day, seasons, physiological parameters and the like. Pulse diagnosis has emerged as a potential technique for Vikriti estimation in Ayurveda. Subsequent to the Prakriti and Vikriti estimation, the doctor obtains a holistic assessment of the individual and prescribes further therapeutic course of action. The therapeutic action combines a variety of approaches such as herbal medicines, panchakarma procedures, food recommendations, Yoga recommendations, meditation recommendations and the like.

However, current methods for both Prakriti and Vikriti estimation in Ayurveda and also various traditional Chinese medicine parameters are highly subjective and may vary depending on doctor's expertise and framework followed for Prakriti and Vikriti estimation. Efficient straight forward approach has to be formulated for the Prakriti estimation, Vikriti estimation and estimation of the various traditional Chinese medicine parameters. Moreover, the pulse diagnosis evaluation process requires various sensor readings. Effective sensor placement and integration of the data from various sensors will help to capture the pulse parameters in real time to estimate traditional medicine system parameters.

Hence, there is a need for a smart wearable device and a method for estimating traditional medicine system parameters and therefore address the aforementioned issues.

### BRIEF DESCRIPTION

In accordance with one embodiment of the disclosure, a wearable device for estimating traditional medicine system parameters is disclosed. The wearable device includes one or more light sources. The one or more light sources is configured to stimulate skin of a patient through light rays. The wearable device also includes one or more sensors. The one or more sensors is configured to capture one or more patient health parameters. The one or more sensors comprises one or more light sensors, a digital image sensor, a magnetic sensor and one or more physiological sensors.

The wearable device also includes a hardware processor. The wearable device also includes a memory coupled to the hardware processor. The memory comprises a set of program instructions in the form of a plurality of subsystems and configured to be executed by the hardware processor.

The plurality of subsystems includes a medical input data collection subsystem. The medical input data collection subsystem is configured to collect patient information and the captured one or more patient health parameters associated with the patient from the one or more sensors, one or more real time inputs from biochemical markers and multi-omics markers and a conversational artificial intelligence questionnaire. The medical input data collection subsystem is also configured to collect blood pulse parameters for ayurveda diagnosis from the one or more sensors. The ayurveda diagnosis comprises pulse rate, pulse rate variability, pulse pressure and pulse transit time. Here pulse morphology refers to pulse shape as measured by change in pulse amplitude with respect to time.

The plurality of subsystems also includes a health status computation subsystem. The health status computation subsystem is configured to apply the collected patient information the captured one or more patient health parameters and the blood pulse parameters associated with the patient on to a trained machine learning model. The health status computation subsystem is also configured to estimate real time set of traditional medicine system parameters based on the results of the trained machine learning model.

The plurality of subsystems also includes a disease identification subsystem. The disease identification subsystem is configured to compare the real time set of traditional medicine system parameters with pre-stored real time set of traditional medicine system parameters. The disease identification subsystem is also configured to identify a disease based on the compared results and based on pre-stored disease database. The disease identification subsystem is also configured to generate a recommendation message to the patient based on the identified disease. In such embodiment, the recommendation message comprises of medical diagnosis of the disease, health parameters, therapeutic interventions, clinical interventions, one or more medical remedies, and treatment plan. The disease identification subsystem is also configured to perform one or more operations based on the generated recommendation message and the patient prior approval.

In accordance with one embodiment of the disclosure, a method for estimating traditional medicine system parameters via a wearable device is disclosed. The method includes collecting patient information and the captured one or more patient health parameters associated with the patient from the one or more sensors, one or more real time inputs from biochemical markers and multi-omics markers and a conversational artificial intelligence questionnaire. The method also includes collecting, by a processor, blood pulse parameters for ayurveda diagnosis from the one or more sensors.

The method also includes applying the collected patient information the captured one or more patient health parameters and the blood pulse parameters associated with the patient on to a trained machine learning model. The method also includes real time set of traditional medicine system parameters based on the results of the trained machine learning model.

The method also includes comparing the real time set of traditional medicine system parameters with pre-stored real time set of traditional medicine system parameters. The method also includes identifying a disease based on the compared results and based on pre-stored disease database.

The method also includes generating a recommendation message to the patient based on the identified disease. The method also includes performing one or more operations based on the generated recommendation message and the patient prior approval.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a schematic representation illustrating a wearable device and associated sensors for estimating traditional medicine system parameters in accordance with an embodiment of the present disclosure;

FIG. 2 is a block diagram illustrating an exemplary computing system for estimating traditional medicine system parameters via the wearable device in accordance with an embodiment of the present disclosure;

FIG. 3 is a schematic representation depicting an exemplary process of estimating Vikriti using smart wearable device, in accordance with an embodiment of the present disclosure;

FIG. 4 is a schematic representation depicting an exemplary process of estimation of prakriti and vikriti combining the inputs of the Ayurveda adaptive questionnaire and smart wearable device, in accordance with an embodiment of the present disclosure;

FIG. 5 is a process flowchart illustrating an exemplary method for estimating traditional medicine system parameters via the wearable device in accordance with an embodiment of the present disclosure;

FIG. 6 A-C are graphical user interface of conversational artificial intelligence based adaptive questionnaire framework designed as per Ayurveda principles for assessing Prakriti baseline of a patient using the wearable device, in accordance with an embodiment of the present disclosure;

FIG. 7 is a schematic representation depicting an exemplary process of estimating Vikriti using wearable device combining conversational AI based symptoms and pulse features, in accordance with an embodiment of the present disclosure,

FIG. 8 is a schematic representation depicting an exemplary process of recommendations, in accordance with an embodiment of the present disclosure;

FIG. 9 is a graphical representation depicting an example of using multi-modal sensor data, accelerometer data and PPG data from the wearable device for estimation of cardiac fitness using static and dynamical digital biomarkers;

FIG. 10 is a graphical representation depicting an example of estimation of cardio health and wellness score for two healthy subjects having the same prakriti using the data collected from wearable device;

FIG. 11 is a graphical representation depicting an example of estimating mental stress from the data collected using wearable device; and

FIG. 12 is a graphical representation depicting an example of estimating vikriti (diabetes score) from the wearable device.

Further, those skilled in the art will appreciate that elements in the figures are illustrated for simplicity and may not have necessarily been drawn to scale. Furthermore, in terms of the construction of the device, one or move components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated online platform, and such further applications of the principles of the disclosure as would normally occur to those skilled in the art are to be construed as being within the scope of the present disclosure.

The terms “comprises”. “comprising”, or any other variations thereof, are intended to cover a non-exclusive inclusion, such that a process or method that comprises a list of steps does not include only those steps but may include other steps not expressly listed or inherent to such a process or method. Similarly, one or more devices or subsystems or elements or structures or components preceded by “comprises . . . , a” does not, without more constraints, preclude the existence of other devices, subsystems, elements, structures, components, additional devices, additional subsystems, additional elements, additional structures or additional components. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a,” “an,” and “the” include plural references unless the context clearly dictates otherwise.

A computer system (standalone, client or server computer system) configured by an application may constitute a “subsystem” that is configured and operated to perform certain operations. In one embodiment, the “subsystem” may be implemented mechanically or electronically, so a subsystem may comprise dedicated circuitry or logic that is permanently configured (within a special-purpose processor) to perform certain operations. In another embodiment, a “subsystem” may also comprise programmable logic or circuitry (as encompassed within a general-purpose processor or other programmable processor) that is temporarily configured by software to perform certain operations.

Accordingly, the term “subsystem” should be understood to encompass a tangible entity, be that an entity that is physically constructed permanently configured (hardwired) or temporarily configured (programmed) to operate in a certain manner and/or to perform certain operations described herein.

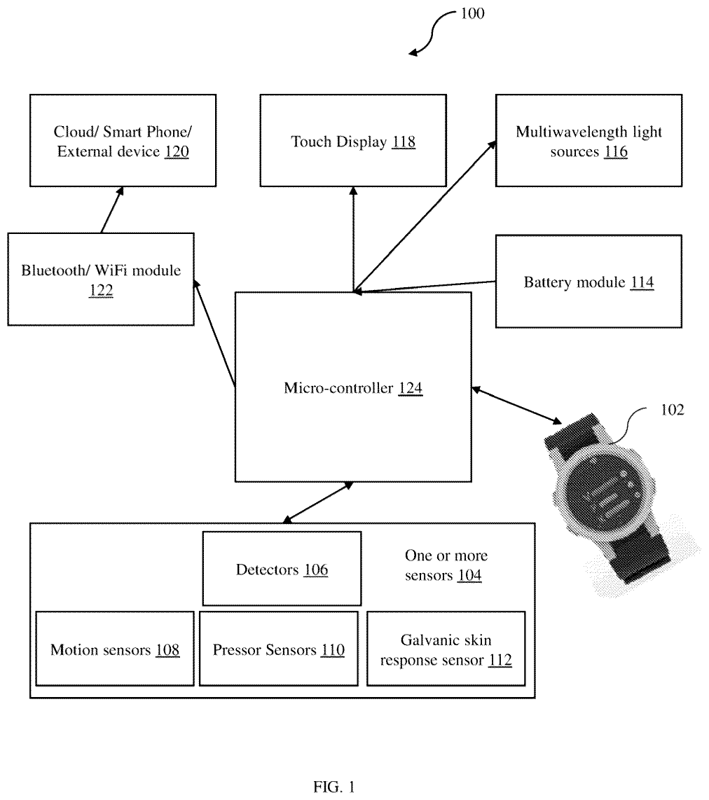

FIG. 1 is a schematic presentation 100 illustrating a wearable device and associated sensors for estimating traditional medicine system parameters in accordance with an embodiment of the present disclosure. Wearable devices are products controlled by electronic components and software that may be incorporated into clothing or worn on the body like accessories. In such embodiment, the wearable devices may include smart watches, smart jackets, and the like. In one embodiment, the set traditional medicine system parameters include Ayurveda variables. Traditional Chinese Medicine variables. Siddha parameters, Unani parameters, and the like. In such embodiment, the set of Ayurveda variables refers to Ayurveda Vata variable, Pitta variable and Kapha variable. The wearable device may be used anywhere on the patient body such as wrist, leg, head, forearm, and the like.

In one exemplary embodiment, the wearable device may be a smart watch 102. The smart watch 102 includes one or more light sources operating in visible (for example: green, red), near infrared wavelengths. The one or more light sources including multiwavelength light sources 116 is configured to excite skin of a patient through light rays. In such embodiment, the skin excitation helps in getting data about underlying patient blood pulsations data. In one embodiment, red and infrared light is used to measure the amount of pulsatile blood flow. In such embodiment, the red light is primarily absorbed by deoxygenated blood and the infrared light is primarily absorbed by oxygenated blood. Pulse waveforms at these wavelengths are captured to extract various traditional medicine parameters such as vega, gati, bala, pulse morphology etc, using machine and deep learning techniques. Further, the ratio of absorption may be measured with the deoxygenated blood and the oxygenated blood absorption result.

The smart watch 102 includes one or more sensors 104 configured to capture one or more patient health parameters. The one or more sensors 104 includes detectors 106, motion sensors 108, pressure sensors 110. Galvanic skin response sensor 112, one or more light sensors, a detector and one or more physiological sensors.

The one or more light sensors is configured to capture patient blood pulse waveform and collect data about underlying patient blood pulsations on application of the light rays. As used herein, the term “pulse wave” is the change in the volume of a blood vessel that occurs when the heart pumps blood. In such embodiment, a pulse sensor monitors this volume change.

The detector includes a digital image sensor, which is configured to capture spatial information of the patient blood pulsations. A magnetic sensor is configured to detect any real time changes in underlying patient blood as the haemoglobin component in blood has magnetic properties. Example of spatial information includes changes in physiological health variables such as heart rate, blood oxygenation, blood pressure of the patient at different locations due to varying geographical conditions such as temperature, humidity, weather, altitude, and the like. The digital image sensor can also be used to analyse how the patient's pulse is changing at various spatial locations (for example near the wrist) of the subject. As used herein, the term “digital image sensor” is a technology used to record electronic images. The one or more physiological sensors is configured to capture physiological parameters and movement parameters of the patient. The one or more physiological sensors includes accelerometer, galvanic sensor, barometer, temperature sensor, and the like. The motion sensors 108 may be used to capture the patient movement parameters.

As used herein, the term “accelerometer” refers to a device that measures the vibration, or acceleration of motion of the patient. As used herein, the term “galvanic sensor” refers to a sensor measuring the electrical conductance of the skin, which varies with its moisture level. As used herein, the term “barometer” refers to a scientific instrument that is used to measure air pressure in a certain environment.

In another embodiment, the anatomic features of the patient as described, am extracted using computer vision techniques. Digital biomarkers (both static markers while the patient is at rest and also dynamic markers that are captured as per the patient transitions from one stale to other) are obtained using various physiological sensors, voice-based techniques. In addition, various biochemical, clinical and multi-omics markers are captured from subject's samples (such as blood, urine, saliva etc.). Multi-omics markers include genetic, transcriptome, epigenome, metagenome, metabolome, glycome, proteome and the like. Digital biomarkers are defined as objective, quantifiable physiological and behavioural data of the patient.

Further, the smart watch 102 is integrated with a battery module 114. The battery module 114 helps in powering the smart watch 102. The smart watch 102 is also integrated with a touch display 118. The touch display 118 helps in viewing the collected patient information, the captured one or more patient health parameters and recommendation messages. The smart watch 102 is also integrated with a Bluetooth or Wi-Fi integrated module 122. The Bluetooth or Wi-Fi integrated module 122 helps in transmitting the collected patient information, the captured one or more patient health parameters and the recommendation messages to an external device or cloud or smart phone 120. In such embodiment, the external device 120 enables storage, analysis and display of the collected data.

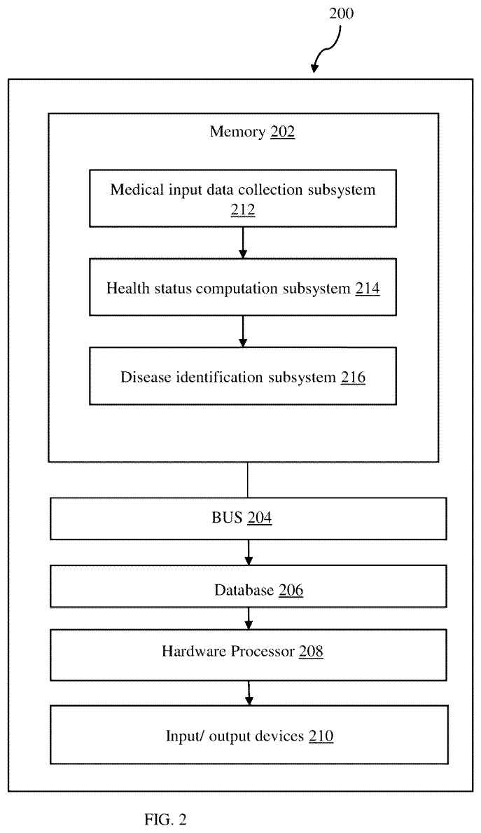

The smart watch 102 also includes a hardware processor (as shown in FIG. 2). The smart watch 102 also includes a memory coupled to the hardware processor. The memory comprises a set of program instructions in the form of a plurality of subsystems and configured to be executed by the hardware processor.

The hardware processor, as used herein, means any type of computational circuit, such as, but not limited to, a microprocessor, a microcontroller 124, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof. In one embodiment, all adjoining above-described wearable device parts are coupled with the hardware processor such as microprocessor for further analysis of data.

FIG. 2 is a block diagram 200 illustrating an exemplary computing system for estimating traditional medicine system parameters via the wearable device in accordance with an embodiment of the present disclosure. The wearable device 102 includes a hardware processor 208. The wearable device 102 also includes a memory 202 coupled to the hardware processor 208. The memory 202 comprises a set of program instructions in the form of a plurality of subsystems and configured to be executed by the hardware processor 208. Input/output (I/O) devices 210 (including but not limited to keyboards, displays, pointing devices, etc.) can be coupled to the wearable device 102 either directly or through intervening I/O controllers.

The memory 202 includes a plurality of subsystems stored in the form of executable program which instructs the hardware processor 208 via bus 204 to perform the method steps. The plurality of subsystems has following subsystems: a medical input data collection subsystem 212, a health status computation subsystem 214 and a disease identification subsystem 216.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor 208.

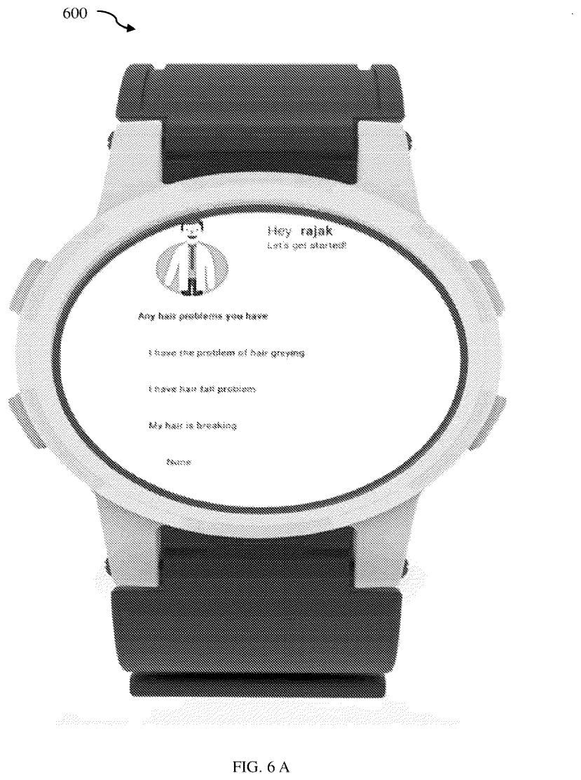

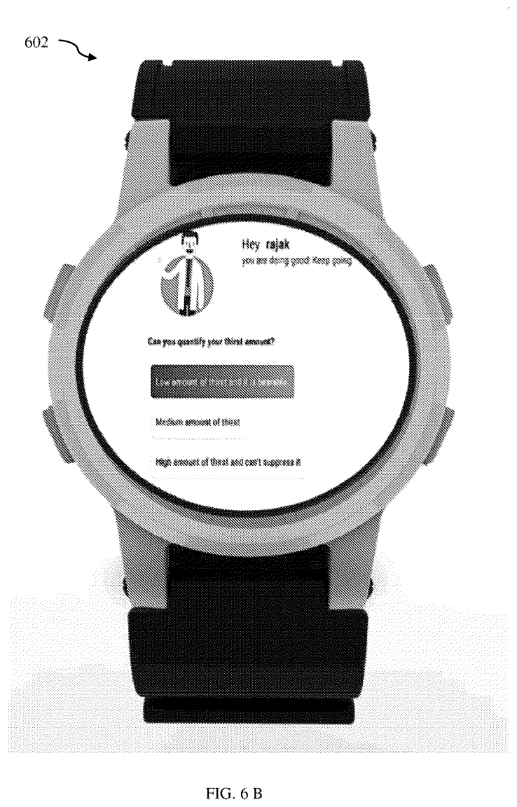

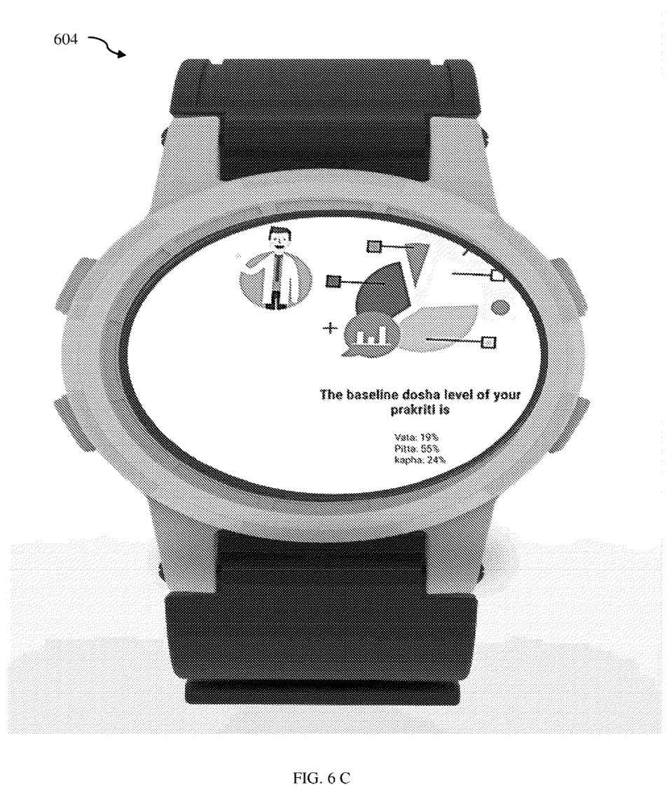

The plurality of subsystems includes a medical input data collection subsystem 212. The medical input data collection subsystem 212 is configured to collect patient information and the captured one or more patient health parameters associated with the patient from the one or more sensors, one or more real time inputs from biochemical markers and multi-omics markers and a conversational artificial intelligence questionnaire (as shown in FIG. 6). The medical input data collection subsystem 212 is also configured to collect blood pulse parameters for ayurveda diagnosis from the one or more sensors. In such embodiment, the ayurveda diagnosis includes pulse rate (vega), pulse rate variability (rhythm), pulse pressure (bala), pulse morphology (gati) and the like.

In such embodiment, the biochemical markers and the multi-omics markers include static markers while the patient is at rest and also dynamic markers that are captured as per the patient transitions from one state to other.

In yet another embodiment, the conversational artificial intelligence questionnaire is used for capturing patient information and phenotypic features. The patient information includes age of the patient, gender of the patient, family history of the patient and the like. The phenotypic features include the anatomic features, physical and physiological features, psychological features, and the like. The conversational artificial intelligence questionnaire is presented to the patient in a digital format such as computers, phones, tablets, smart watches, and the like. In such embodiment, the conversational artificial intelligence questionnaire may have both text and voice format. The conversational artificial intelligence questionnaire also has multilingual support. In such embodiment, the computing system 200 optimizes the number and (or) type of questions needed to assess the patient based on replies given to the previous questions until a desired accuracy level is reached.

The questionnaire may include genetic history of various diseases in the family, death due to heart attacks, behaviour inputs such as type of food eaten, increase in weight over the years, exercise frequency and clinical symptoms such as pain in the chest, dizziness after exercise, and the like. In such embodiment, the collected readings are combined with health information of the patient.

The plurality of subsystems also includes a health status computation subsystem 214. The health status computation subsystem 214 is configured to apply the collected patient information the captured one or more patient health parameters and the blood pulse parameters associated with the patient on to a trained machine learning model. The machine learning model using algorithms such as logistic regression, random forest, support vector machine classifier, deep learning models or a combination thereof. In one embodiment, the collected patient information includes the patient personal details that are captured during registration process.

In such embodiment, the health status computation subsystem 214 applies the obtained responses for the set of adaptive and conversational artificial intelligence-based questionnaire, the obtained one or more real time patient information, obtained one or more real time spatiotemporal information data and the obtained one or more real time inputs from biochemical markers and multi-omics markers onto the trained machine learning model. As used herein, the term “Spatiotemporal, or spatial temporal” is used in data analysis when data is collected across both space and time.

The trained machine learning model use algorithms such as logistic regression, random forests, support vector machines, nearest neighbour clustering, boosted generalized additive models, neural networks, and the like, or may include a combination of the above algorithms. In one embodiment, the conversational artificial intelligence questionnaire is converted into categorical variables in a matrix form and then applied to the trained machine learning model. In such embodiment, the other forms of collected data is directly applied to the trained machine learning model.

The health status computation subsystem 214 is also configured to estimate real time set of traditional medicine system parameters based on the results of the trained machine learning model. In one embodiment, the set traditional medicine system parameters include Ayurveda variables, Traditional Chinese Medicine variables. Siddha parameters, Unani parameters, and the like. In such embodiment, the set of Ayurveda variables refers to Ayurveda Vata variable, Pitta variable and Kapha variable. In one embodiment, the set of Traditional Chinese Medicine variables refer to Traditional Chinese Medicine Cun variable. Guan variable and Chi variable. In one specific embodiment, the changes in Vata variable, the Pitta variable and the Kapha variable is being used to estimate Vikriti. Prakriti estimation is typically done using a questionnaire. Traditional medicine systems parameters may also include dhatus effected due to Vikriti.

The plurality of subsystems also includes a disease identification subsystem 216. The disease identification subsystem 216 is configured to the real time set of traditional medicine system parameters with pre-stored real time set of traditional medicine system parameters. The disease identification subsystem 216 is also configured to identify a disease based on the compared results and based on pre-stored disease database. In one embodiment, the pre-stored disease database is stored in a database 206.

The disease identification subsystem 216 is also configured to generate a recommendation message to the patient based on the identified disease. In such embodiment, the recommendation message includes of medical diagnosis of the disease, health parameters, therapeutic interventions, clinical interventions, one or more medical remedies, and treatment plan.

Furthermore, the disease identification subsystem 216 is also configured to perform one or more operations based on the generated recommendation message and the patient prior approval. In such embodiment, the one or more operations includes generating alerts for the patient representatives, generating alerts for medical representatives, generating new treatment plan and generating new diet plan. For example, if a recommendation message generates immediate clinical interventions for any patient, the disease identification subsystem 216 is configured to alert the medical representatives of the patient in real time through emails, messages or a call.

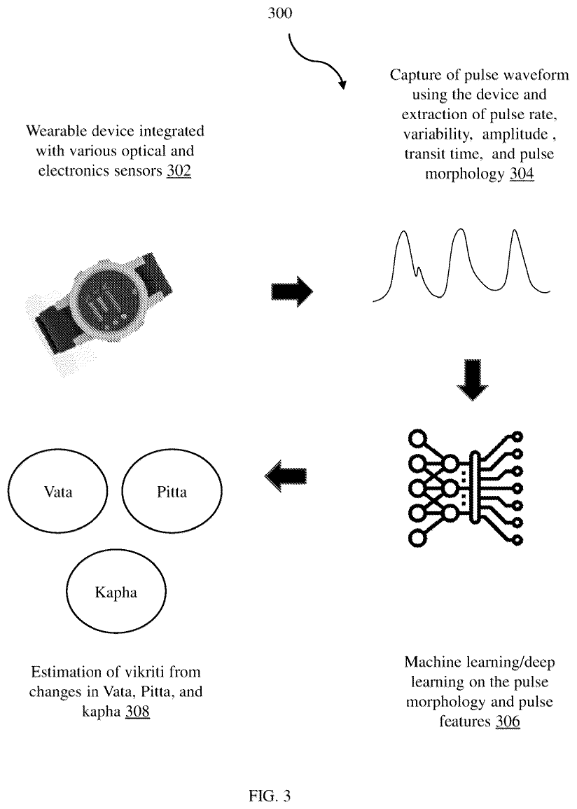

FIG. 3 is a schematic representation depicting an exemplary process 300 of estimating Vikriti using smart wearable device, in accordance with an embodiment of the present disclosure. The wearable device 302 is integrated with various optical and electronic sensors. In one particular embodiment, the wearable device 302 may include a smart watch. In such embodiment, the wearable device 302 captures patient information and one or more patient health parameters.

The wearable device 302 with the optical and electronic sensors captures pulse waveform 304 and extracts pulse features such as pulse rate, variability, amplitude, transit time, and pulse morphology. Further, the above extracted pulse features are applied to trained machine learning model 306. The trained machine learning model 306 helps in determining vikriti from the changes in Vata, Pitta and Kapha, dhatus (as defined in Ayurveda such as blood, muscle, fat, plasma, bone, bone marrow etc.) effected, and the like.

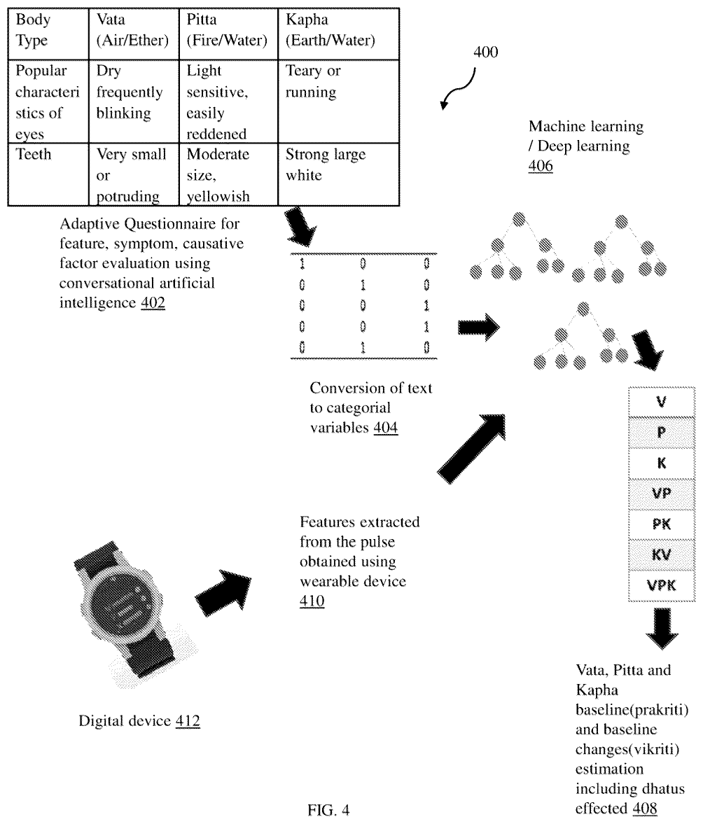

FIG. 4 is a schematic representation 400 depicting an exemplary process of estimation of prakriti and vikriti combining the inputs of the Ayurveda adaptive questionnaire and smart wearable device, in accordance with an embodiment of the present disclosure.

In such exemplary embodiment, these adaptive questionnaires 402 comprises features, symptom-based questions in text format. As used herein, the term “feature” refers to different parameters of the patient, such as physical, psychological, anatomical, physiological, for example, skin colour, digestion, thinking, walking and the like. In such embodiment, the adaptive questionnaires 402 comprises questions related to phenotypic features such as walking style, anger quality, digestion habits and disease symptoms such as pain in certain part of the body such as eye, teeth, and the like, sleep disorders, changed body weight, excessive thirst or urination and the like. These adaptive questionnaires 402 are outputted to the patient on his device for obtaining a response for each of these adaptive questionnaires 402.

The text of the questionnaire is converted into categorical variables 404, such as 0s, 1s and 2s. Furthermore, trained machine learning models 406 are used onto each of these categorical variables 404 to estimate the set of Ayurveda variable. Vata. Pitta and Kapha baseline, baselines changes in Vata, Pitta and Kapha and dhatus effected 408. In one exemplary embodiment, a random forest tree machine learning algorithm may be used.

Simultaneously, inputs from wearable device 412 are collected. Then, features 410 are extracted from pulse obtained using the wearable device 412. In such embodiment, the digital device 412 may be a wearable device, such as a smartwatch. These extracted features 410 includes pulse rate, pulse rate variability, nonlinear measures of pulse rate variations, pulse pressure, pulse morphology, and the like, are also fed as additional input to the trained machine learning models 406 to estimate the estimate the set of Ayurveda variable. Vata. Pitta and Kapha baseline, baselines changes in Vata. Pitta and Kapha and dhatus effected 408.

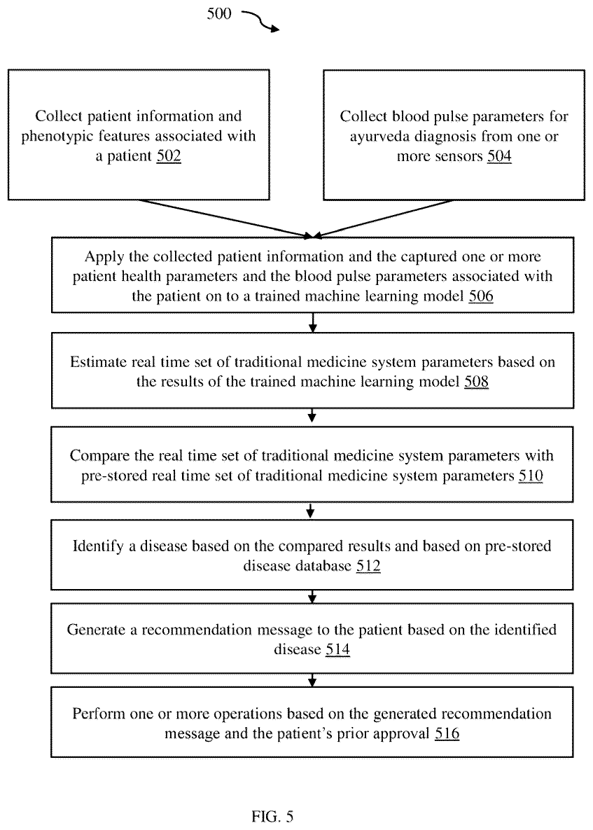

FIG. 5 is a process flowchart illustrating an exemplary method 500 for estimating traditional medicine system parameters via the wearable device in accordance with an embodiment of the present disclosure. In step 502, patient information and one or more patient health parameters associated with the patient are collected from one or more sensors, one or more inputs from biochemical markers and multi-omics markers and a conversational artificial intelligence questionnaire. In one aspect of the present embodiment, the patient information and one or more patient health parameters associated with the patient are collected by a medical input data collection subsystem 212. In such embodiment, the digital biomarkers, biochemical markers and the multi-omics markers include static markers while the patient is at rest and also dynamic markers that are captured as per the patient transitions from one state to other.

The captured one or more patient health parameters includes patient phenotypic features of the patient. The patient phenotypic features comprise anatomic features, physical, physiological features, psychological features.

In step 504, blood pulse parameters are collected for ayurveda diagnosis from the one or more sensors. In such embodiment, the ayurveda diagnosis comprises pulse rate, pulse rate variability, pulse pressure, pulse transit time and pulse morphology.

In step 506, the collected patient information and the captured one or more patient health parameters and the blood pulse parameters associated with the patient are applied on to a trained machine learning model. In one aspect of the present embodiment, the collected patient information and the captured one or more patient health parameters and the blood pulse parameters associated with the patient are applied by the health status computation subsystem 214.

In step 508 real time set of traditional medicine system parameters are estimated based on the results of the trained machine learning model, in one aspect of the present embodiment, the real time set of traditional medicine system parameters are estimated based on the results of the trained machine learning model by the health status computation subsystem 214.

In step 510, the real time set of traditional medicine system parameters is compared with pre-stored real time set of traditional medicine system parameter, in one aspect of the present embodiment, the real time set of traditional medicine system parameters is compared with pre-stored real time set of traditional medicine system parameter by a disease identification subsystem 216.

In step 512, a disease is identified based on the compared results and based on pre-stored disease database. In one aspect of the present embodiment, the disease is identified by the disease identification subsystem 216. The disease includes diabetes, cardiovascular diseases and gastro-intestinal diseases.

In step 514, a recommendation message is generated to the patient based on the identified disease. In one aspect of the present embodiment, the recommendation message is generated by the disease identification subsystem 216. The recommendation message comprises of medical diagnosis of the disease, health parameters, therapeutic interventions, clinical interventions, one or more medical remedies, and treatment plan.

In step 516, one or more operations are performed based on the generated recommendation message and the patient prior approval. In one aspect of the present embodiment, the one or more operations are performed by the disease identification subsystem 216. The one or more operations comprises generating alerts for the patient representatives, generating alerts for medical representatives, generating new treatment plan and generating new diet plan.

FIG. 6 A-C are graphical user interface of conversational artificial intelligence based adaptive questionnaire framework designed as per Ayurveda principles for assessing Prakriti baseline of a patient 602, 604, 606 and 608, in accordance with an embodiment of the present disclosure. Each of the question probes the Vata, Pitta or Kapha characteristics of the person based on the phenotypic features. The weights for each of the questions are pre-assigned based on the clinical knowledge or from the machine learning algorithm. At the end of each question/set of questions, the scores are calculated for Vata, Pita and Kapha.

The questions are presented to the subjected till the successive difference in the scores from a question/set of questions is less than a pre-set threshold value. This conversational artificial intelligence based adaptive questionnaire saves significant time in assessing the prakriti of any person while achieving the desired accuracy. Similar, conversational artificial intelligence-based framework may also be used for assessing the Vikriti (diseased state) of a person. Some of the questions pertaining to the symptoms are “Heart burn,” “Pain in the head,” “frequent urination” and the like.

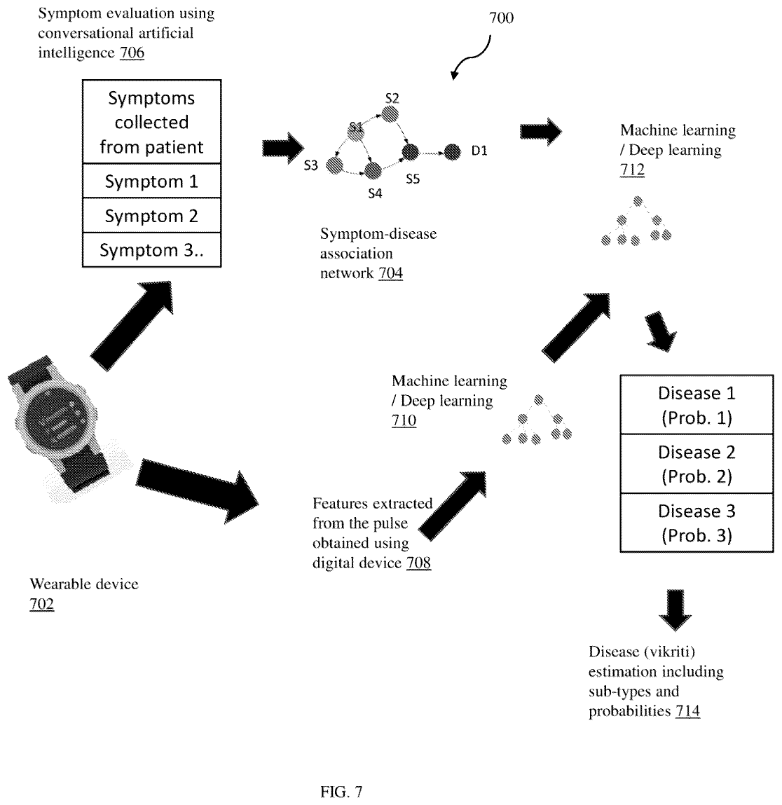

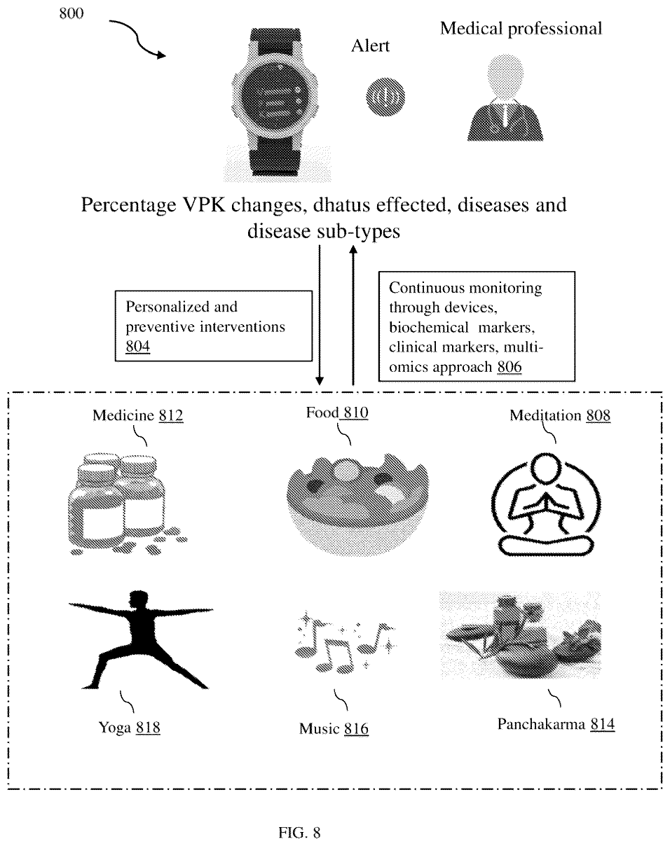

FIG. 7 is a schematic representation depicting an exemplary process 700 of estimating Vikriti using wearable device combining conversational AI based symptoms and pulse features, in accordance with an embodiment of the present disclosure. Wearable device 702 collects symptom evaluation using conversational artificial intelligence 706 and other features 708. Artificial intelligence machine leering 712 is applied to understand disease 1 or disease 2 714 is associated with the patient.

FIG. 5 is a schematic representation depicting an exemplary process of recommendations, in accordance with an embodiment of the present disclosure. The recommendations may include Personalized and preventive interventions 804. Such recommendations may include food 810, medicine 812, meditations 808, panchakarma 814, yoga 818 and music 816. Such recommendations are alerted to the patient by the medical professions by looking into percentage changes, dhatus effected, diseases and disease sub-types.

The present disclosure reveals an artificial intelligence integrated wearable device 102 for estimating traditional medicine system parameters. The present wearable device 102 provides a continuous objective decision-making system. The wearable device 102 is linked to a smart phone and consists of an explainable AI configured to explain decision or estimation pathway. The explainable AI will give information on what features have contributed to the baseline and disease determination and relative importance of each feature to the decision. The features can include phenotypic features, digital biomarkers captured from the pulse, biochemical, clinical, multi-omics parameters and the like.

The wearable device 102 uses inputs from the adaptive questionnaire using conversational artificial intelligence for Prakriti and Vikriti estimation. The wearable device 102 is wearable and hence captures key pulse and physiological parameters continuously. Machine and deep learning techniques provide highly accurate decision. The present disclosed system 200 overcomes the challenge of need of an expert to read the pulse and determine the Vikriti. The present disclosed system 200 continuously monitors the blood pulse while correcting for all physical factors using non-invasive methods. In addition, the wearable device collects key information from both static and dynamical markers as the patient transitions from one state to other (for example from sitting to standing, walking up the stairs, awake from sleep and the like.)

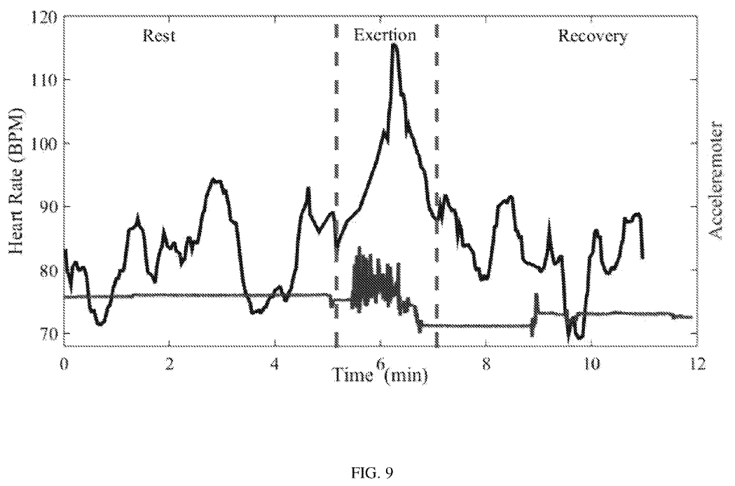

FIG. 9 is a graphical representation depicting an example of using multi-modal sensor data i.e., accelerometer data and PPG data from the wearable device for estimation of cardiac fitness using static and dynamical digital biomarkers. A subject's prakriti is first assessed using the conversational AI framework as depicted in FIG. 6. The prakriti is determined as Pitta-Kapha. The wearable device continuously obtains the accelerometer and PPG data. Accelerometer data provides an estimation of physical exertion (sitting to standing and the like.) which can be used to understand the changes in digital biomarkers such as heart rate as the subject transitions from one state to other. Large changes in these metrics and long recovery times to the values as seen in the rest condition after the changes have occurred correspond to low cardiac fitness. In addition, while estimating the cardiac fitness prakriti of the person is taken in account in addition to age, weight, gender and the like.

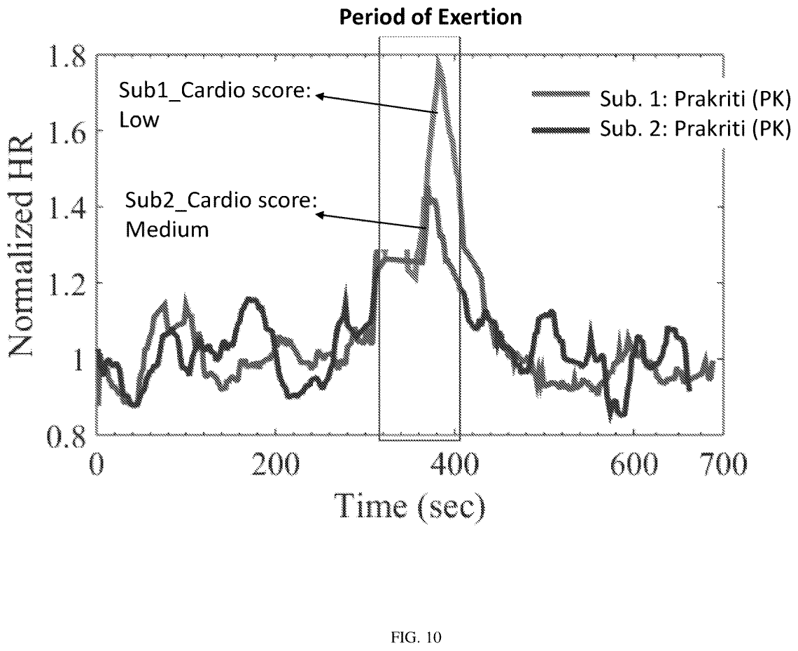

FIG. 10 is a graphical representation depicting an example of estimation of cardio health and wellness score for two healthy subjects having the same prakriti using the data collected from wearable device. The two healthy subjects prakriti is first assessed using the conversational AI framework as depicted in FIG. 6. Both the subjects have the same prakriti (mixed prakriti designated as Pitta-Kapha). The subjects photoplethysmograph (PPG) signal is collected using the green light sources and a silicon detector integrated into the wearable device. The subject's data is collected first at rest condition and subsequently the subjects have undergone physical exertion by climbing the stairs. Data is also collected after the period of exertion is over. The PPG data collected during the entire duration is further processed for extracting the heart rate changes during the entire process. Since the subjects resting heart rate is different, the entire data is normalized with respect to the heart rate at the rest condition. As seen from the figure, during the physical exertion, the heart rate increases. Smaller change in the heart rate for a given exertion level corresponds to a good cardio fitness while a large change in the heart rate for a given exertion level corresponds to poor cardio fitness. For this experiment, a cardio score is designed such that the maximum change of normalized heart rate greater than 1.6 corresponds to low score, between 1.3 to 1.6 corresponds to medium score and less than 1.3 corresponds to high score. Accordingly, subject 1 has a low cardio score while subject two has medium cardio score. In summary, the example demonstrates the ability of wearable device to estimate the cardiac health and wellness score using static and dynamic changes in heart rate. Other heart rate variability markers (root mean square of successive differences between normal heartbeats, Standard deviation of heartbeat intervals) can also be used to design a composite health and wellness score.

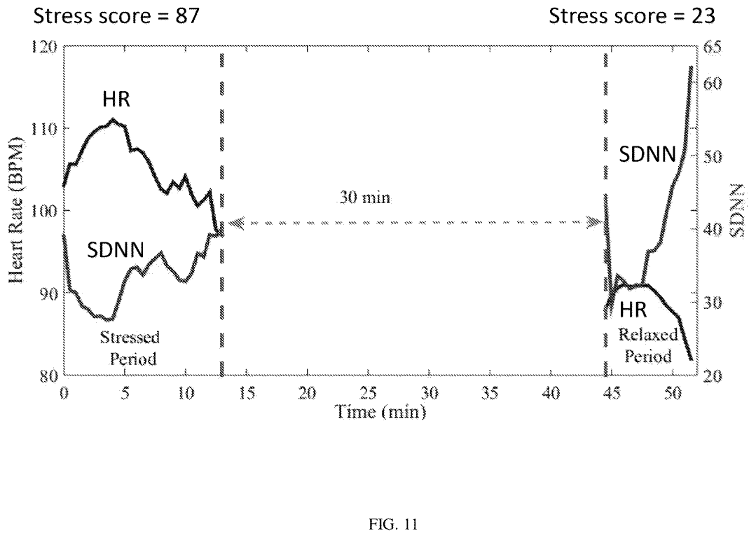

FIG. 11 is a graphical representation depicting an example of estimating mental stress from the data collected using wearable device. A subject's prakriti is first assessed using the conversational AI framework depicted as in FIG. 6. The prakriti is determined is mixed prakriti: Vata-Pitta-Kapha. Subsequently, the wearable device continuously records the PPG data. Heart rate and heart rate variability metrics such as SDNN (Successive deviation of the heartbeat intervals) are calculated for the subject. To estimate the mental stress, various confounding factors are corrected for such as the person's prakriti (as it is known that vata, pitta and kapha prakriti people respond differently to mental stress), time of the day, season, weekday/weekend and the like. Finally, a machine learning algorithm based on Random forest is used considering the heart rate, heart rate variability metrics and also various confounding factors to estimate and display a real time stress score. Based on the calculated stress score, a corrective action such as meditation, listening to soothing music is suggested. In case of extreme stress, an alert to the medical professional is also generated.

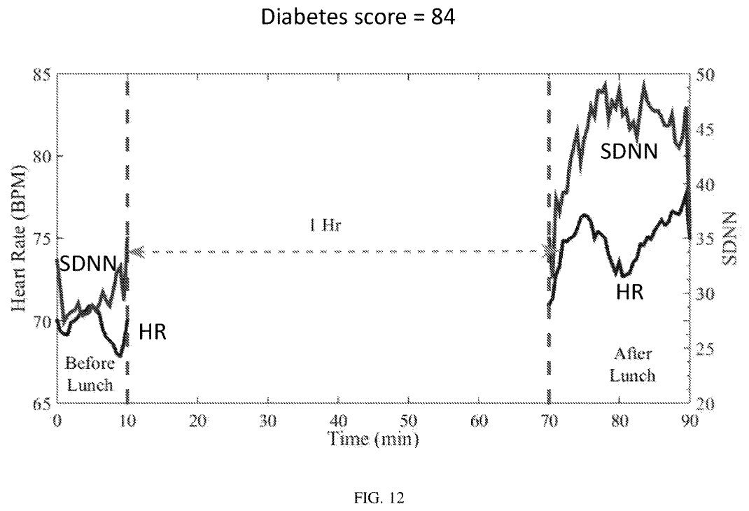

FIG. 12 is a graphical representation depicting an example of estimating vikriti (diabetes score) from the wearable device A subject's prakriti is first assessed using the conversational AI framework depicted in FIG. 6. The subjects prakriti is determined as mixed prakriti Pitta-Vata. Subsequently, the wearable device continuously records the PPG data before and after the subject takes food. Heart rate, heart rate variability (depicted using SDNN) are calculated for the subject before and after food. To estimate the diabetes score various confounding factors are considered such as the person's prakriti (as it is known that vata, pitta and kapha prakriti people have different predisposition to diabetes), time of the day, season, type of food eaten etc. Finally, a machine learning algorithm based on random forest is used to estimate diabetes score. Based on the calculated diabetes score, a corrective action such as avoiding certain foods, yoga, meditation is suggested. In case of high diabetes risk score, an alert to the medical professional is also generated. In addition, pulse morphology can be included in the estimation and deep learning algorithms such as neural networks can be used to predict the diabetes risk score.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, and the like. The functions performed by various modules described herein may be implemented in other modules or combinations of other modules. For the purposes of this description, a computer-usable or computer readable medium can be any apparatus that can comprise, store, communicate, propagate, or transport the program for use by or in connection with the instruction execution system, apparatus, or device.

The medium can be an electronic, magnetic, optical, electromagnetic, infrared, or semiconductor system (or apparatus or device) or a propagation medium. Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD.

Input/output (I/O) devices (as shown in FIG. 1) (including but not limited to keyboards, displays, pointing devices, etc.) can be coupled to the system either directly or through intervening I/O controllers. Network adapters may also be coupled to the system to enable the data processing system to become coupled to other data processing systems or remote printers or storage devices through intervening private or public networks. Modems, cable modem and Ethernet cards are just a few of the currently available types of network adapters.

A representative hardware environment for practicing the embodiments may include a hardware configuration of an information handling/computer system in accordance with the embodiments herein. The system herein comprises at least one processor or central processing unit (CPU). The system may additionally include a GPU. The CPUs are interconnected via system bus to various devices such as a random-access memory (RAM), read-only memory (ROM), and an input/output (I/O) adapter. The I/O adapter can connect to peripheral devices, such as disk units and tape drives, or other program storage devices that are readable by the system. The system can read the inventive instructions on the program storage devices and follow these instructions to execute the methodology of the embodiments herein.

The system further includes a user interface adapter that connects a keyboard, mouse, speaker, microphone, and/or other user interface devices such as a touch screen device (not shown) to the bus to gather user input. Additionally, a communication adapter connects the bus to a data processing network, and a display adapter connects the bus to a display device which may be embodied as an output device such as a monitor, printer, or transmitter, for example.

A description of an embodiment with several components in communication with each other does not imply that all such components am required. On the contrary, a variety of optional components are described to illustrate the wide variety of embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed.

Finally, the language used in the specification has been principally selected for readability and instructional purposes, and it may not have been selected to delineate or circumscribe the inventive subject matter. It is therefore intended that the scope of the invention be limited not by this detailed description, but by any claims that issue on an application based here on. Accordingly, the embodiments of the present invention are intended to be illustrative, but not limiting, of the scope of the invention, which is set forth in the following claims.

The figures and the foregoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, order of processes described herein may be changed and are not limited to the manner described herein. Moreover, the actions of any flow diagram need not be implemented in the order shown; nor do all of the acts need to be necessarily performed. Also, those acts that are not dependant on other acts may be performed in parallel with the other acts. The scope of embodiments is by no means limited by these specific examples.

1. A wearable device for estimating traditional medicine system parameters, the wearable device comprising:
one or more light sources configured to stimulate skin of a patient through light rays;
one or more sensors configured to capture one or more patient health parameters, wherein one or more sensors comprises one or more light sensors, a detector, and one or more physiological sensors;
a hardware processor; and
a memory coupled to the hardware processor, wherein the memory comprises a set of program instructions in the form of a plurality of subsystems, configured to be executed by the hardware processor, wherein the plurality of subsystems comprises:
a medical input data collection subsystem configured to
collect patient information and the captured one or more patient health parameters associated with the patient from the one or more sensors, one or more inputs from biochemical markers and multi-omics markers and a conversational artificial intelligence questionnaire, wherein the digital biomarkers, biochemical markers and the multi-omics markers include static markers while the patient is at rest and also dynamic markers that are captured as per the patient transitions from one state to other; and
collect blood pulse parameters for ayurveda diagnosis from the one or more sensors, wherein the ayurveda diagnosis comprises pulse rate, pulse rate variability, pulse pressure, pulse transit time, pulse morphology;
a health status computation subsystem configured to:
apply the collected patient information the captured one or more patient health parameters and the blood pulse parameters associated with the patient on to a trained machine learning model;
estimate real time set of traditional medicine system parameters based on the results of the trained machine learning model;
a disease identification subsystem configured to:
compare the real time set of traditional medicine system parameters with pre-stored real time set of traditional medicine system parameters;
identify a disease based on the compared results and based on pre-stored disease database;
generate a recommendation message to the patient based on the identified disease, wherein the recommendation message comprises of medical diagnosis of the disease, health parameters, therapeutic interventions, clinical interventions, one or more medical remedies, and treatment plan; and
perform one or more operations based on the generated recommendation message and the patient prior approval, wherein the one or more operations comprises generating alerts for the patient representatives, generating alerts for medical representatives, generating new treatment plan and generating new diet plan.

2. The wearable device as claimed in claim 1, wherein the one or more light sensors is configured to capture patient blood pulse waveform and collect data about underlying patient blood pulsations on application of the light rays.

3. The wearable device as claimed in claim 1, wherein the detector comprises a digital image sensor, wherein the digital sensor is configured to capture spatial information of the patient blood pulsations.

4. The wearable device as claimed in claim 1, wherein the one or more physiological sensors comprises a magnetic sensor, wherein the magnetic sensor is configured to detect any real time changes in underlying patient blood.

5. The wearable device as claimed in claim 1, wherein the one or more physiological sensors configured to capture physiological parameters and movement parameters of the patient, wherein the one or more physiological sensors comprises accelerometer, galvanic sensor, barometer and temperature sensor.

6. The wearable device as claimed in claim 1, wherein the identified diseases comprise diabetes, cardiovascular diseases and gastro-intestinal diseases.

7. The wearable device as claimed in claim 1, wherein the captured one or more patient health parameters comprises patient phenotypic features of the patient, wherein the patient phenotypic features comprise anatomic features, physical, physiological features, psychological features.

8. The wearable device as claimed in claim 1, further comprising a battery module (114) configured to the wearable device.

9. The wearable device as claimed in claim 1, further comprising a touch display configured to view the collected patient information, the captured one or more patient health parameters and the recommendation messages.

10. The wearable device as claimed in claim 1, further comprising a Bluetooth or Wi-Fi integrated module configured to enable transmit of the collected patient information, the captured one or more patient health parameters and the recommendation messages to an external device.

11. The wearable device as claimed in claim 1, wherein to estimate the real time set of traditional medicine system parameters based on the results of the trained machine learning model, the health status computation subsystem and disease identification system configured to estimate dosha imbalance and dhatus effected.

12. The wearable device as claimed in claim 1, wherein to estimate the real time set of traditional chinese medicine system parameters based on the results of the of the trained machine learning model, the health status computation subsystem and disease identification system configured to estimate baseline cun, guan and chi and imbalance in cun, guan and chi.

13. A method for estimating traditional medicine system parameters via a wearable device, the method comprises:
collecting, by a processor, patient information and one or more patient health parameters associated with the patient from the one or more sensors, one or more inputs from biochemical markers and multi-omics markers and a conversational artificial intelligence questionnaire, wherein the biochemical markers and the multi-omics markers include static markers while the patient is at rest and also dynamic markers that are captured as per the patient transitions from one state to other;
collecting, by a processor, blood pulse parameters for ayurveda diagnosis from the one or more sensors, wherein the ayurveda diagnosis comprises pulse rate, pulse rate variability, pulse pressure and pulse transit time;
applying, by the processor, the collected patient information the captured one or more patient health parameters and the blood pulse parameters associated with the patient on to a trained machine learning model;
estimating, by the processor, real time set of traditional medicine system parameters based on the results of the trained machine learning model;
comparing, by the processor, the real time set of traditional medicine system parameters with pre-stored real time set of traditional medicine system parameters;
identifying, by the processor, a disease based on the compared results and based on pre-stored disease database;
generating, by the processor, a recommendation message to the patient based on the identified disease, wherein the recommendation message comprises of medical diagnosis of the disease, health parameters, therapeutic interventions, clinical interventions, one or more medical remedies, and treatment plan; and
performing, by the processor, one or more operations based on the generated recommendation message and the patient prior approval, wherein the one or more operations comprises generating alerts for the patient representatives, generating alerts for medical representatives, generating new treatment plan and generating new diet plan.

14. The method as claimed in claim 13, wherein identifying, by the processor, of the disease comprises diabetes, cardiovascular diseases and gastro-intestinal diseases.

15. The method as claimed in claim 13, wherein applying, by the processor, the captured one or more patient health parameters comprises patient phenotypic features of the patient, wherein the patient phenotypic features comprise anatomic features, physical, physiological features, psychological features.
